(19) United States
(12) Patent Application Publication
(10) Pub. No.: US 2006/0089328 A1
(43) Pub. Date: Apr. 27, 2006

(54) Ready-to-use gemcitabine solutions

(76) Inventors: Edgar Schridde, Hannover (DE); Bernd Merbach, Burgwedel (DE); Stefan-Peter Gimmel, Hannover (DE)

Correspondence Address: KENYON & KENYON LLP, ONE BROADWAY, NEW YORK, NY, 10004, US

(21) Appl. No.: 10/971,659

(22) Filed: Oct. 22, 2004

(51) Int. Cl. A61K 31/7072

(52) U.S. Cl. 514/49

(57) ABSTRACT

Provided are ready-to-use pharmaceutical compositions in the form of gemcitabine solutions.

### FIELD OF THE INVENTION

The present invention relates to pharmaceutical preparations containing gemcitabine in the form of ready-to-use solutions.

### BACKGROUND OF THE INVENTION

Gemcitabine (2′-deoxy-2′,2′-difluorocytidine; 1-(4-amino-2-oxo-1H-pyrimidin-1-yl)-2-deoxy-2,2-difluororibose; dFdC; CAS No. 95058-81-4; C₉H₁₁F₂N₃O₄, Mᵣ 263.2) is an officially monographed substance in the Pharmacopoeia (Official Monographs, USP 27, 1st Supplement USP-NF, page 3060-61, relating to “Gemcitabine Hydrochloride” and “Gemcitabine for Injection”). Gemcitabine has the following chemical structure:

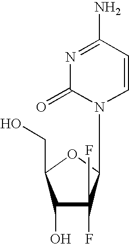

Gemcitabine is used in the treatment of viral infections or immunosuppressive therapy of autoimmune diseases. Gemcitabine was first disclosed in U.S. Pat. No. 4,526,988. U.S. Pat. No. 5,464,826 discloses the antineoplastic effectiveness of gemcitabine. Gemcitabine can be used therapeutically by itself, or in combination with other cytostatic drugs, such as with cisplatin in the treatment of local, advanced, or metastasized non-small-cell bronchial carcinoma, as well as advanced adenocarcinoma or cystadenocarcinoma of the exocrine pancreas.

The recommended dose for gemcitabine therapy is 1 g/m² of body surface area. As other nucleoside analogs, gemcitabine can also be used cytostatically in the therapeutic treatment of the most varied types of cancer, such as lymphatic or myeloid leukemia. Here, the administration of gemcitabine to treat the most varied cancer conditions is effected intravenously, in which case the active substance must be in the form of a solution.

The gemcitabine preparations required for parenteral administration are currently available only in the form of lyophilisates (GEMZAR®), which must be reconstituted before administration to the patient. However, the use of such freeze-dried preparations has considerable disadvantages. First of all, the process of preparing these lyophilisates is complicated and costly. Secondly, reconstitution requires additional working steps and undesirable risks for the personnel involved. In particular, reconstitution of drug solutions from a dry substance can result in what has been called the “spray-back effect,” which may result in further contamination and risk to the personnel. Accordingly, in both the preparation of the lyophilisate and its reconstitution, any contamination of the personnel or stock with the highly effective cytostatic must be avoided. Furthermore, other errors in the handling of these lyophilisates can lead to serious problems such as deviation in the concentration of the active substance, or microbial contamination of the solution prepared from the lyophilisate.

Gemcitabine solutions reconstituted from lyophilisates are unstable because they are subject to degradation of the active substance during storage. Degradation leads to deviation in the concentration of the active substance and contamination by the decomposition products of the gemcitabine solution. Contaminated gemcitabine solutions are undesirable for treatment purposes due to potential risks to the patient.

The object of the present invention is to provide stable, ready-to-use gemcitabine solutions without the above-discussed risks and drawbacks of the known dosage forms and solutions prepared according from them.

### SUMMARY OF THE INVENTION

One aspect of the invention is directed towards an injectable pharmaceutical composition comprising a solution of gemcitabine having a pH of about 3.5 to about 10 and a gemcitabine concentration of about 0.5 mg/ml to about 16 mg/ml in a solvent.

In a preferred embodiment, the pharmaceutical composition is not reconstituted from a solid substance at least 24 hours before being administered to a mammal. Preferably, the pharmaceutical composition is not reconstituted from a solid substance at least 72 hours before being administered to a mammal.

In a preferred embodiment, the gemcitabine concentration is about 5 mg/ml to about 12 mg/ml. Preferably, the gemcitabine concentration is about 10 mg/ml.

In a preferred embodiment, the solution is prepared from a free gemcitabine base.

In a preferred embodiment, the solution is prepared from a physiologically-acceptable acid addition salt of gemcitabine base. Preferably, the physiologically-acceptable acid addition salt is gemcitabine hydrochloride.

In a preferred embodiment, the solvent is selected from the group consisting of water, ethyl alcohol, polyethylene glycol 200-600, and propylene glycol. Preferably, the solvent is water.

In a preferred embodiment, the solution has a pH of about 7 to about 9. Preferably, the solution has a pH of about 7.8 to about 8.2. Also preferably, the solution has a pH of about 7.35 to about 7.55.

In a preferred embodiment, the pH is adjusted by combining or converting gemcitabine base with/to a physiologically-acceptable acid addition salt thereof. Preferably, the physiologically-acceptable acid addition salt is gemcitabine hydrochloride.

In a preferred embodiment, the pH is adjusted with at least one physiologically-acceptable acid. Preferably, the acid is selected from the group consisting of hydrochloric acid, phosphoric acid, sulfuric acid, acetic acid, lactic acid, citric acid, methanesulfonic acid, and ethanesulfonic acid. More preferably, the acid is hydrochloric acid.

In a preferred embodiment, the pH is adjusted with at least one physiologically-acceptable base. Preferably, the base is selected from the group consisting of sodium hydroxide, potassium hydroxide, calcium hydroxide, and magnesium hydroxide. More preferably, the base is sodium hydroxide.

In a preferred embodiment, the solution further comprises a buffer. Preferably, at least one functional group of the buffer acid or buffer base is within the pK range about 2.5 to about 11. More preferably, the buffer is prepared from a reagent selected from the group consisting of tris(hydroxymethyl)-aminomethane, 1-deoxy-(methylamino)-D-glucitol, sodium acetate, disodium hydrogen phosphate, and mixtures thereof.

In a preferred embodiment, the buffer is in the amount of about 0.001 g to about 100 g buffer component per 1 g of gemcitabine. Preferably, the buffer is in the amount of about 0.05 g to about 20 g buffer component per 1 g of gemcitabine. More preferably, the buffer is in the amount of about 0.1 g to about 10 g buffer component per 1 g of gemcitabine.

In another preferred embodiment, the pharmaceutical composition further comprises at least one tonic adjuvant, preservative, antioxidant, or mixtures thereof.

In a preferred embodiment, the pharmaceutical composition comprises a solution having a gemcitabine concentration of about 10 mg/ml, a pH of about 8, and water as the solvent.

In a preferred embodiment, the solution remains free of visible particles after storage at a temperature of about 25° C. for about 18 months.

Another aspect of the invention is directed towards a package for distribution comprising the pharmaceutical composition according the invention, wherein the solution is ready for administration to a mammal without further solubilization of gemcitabine.

Another aspect of the invention is directed towards a method of treating tumors comprising administering the pharmaceutical composition according to the invention to a mammal in need thereof.

In another aspect, the invention is directed towards an injectable pharmaceutical composition comprising a solution of gemcitabine, a pH of about 3.5 to about 10, and a gemcitabine concentration of about 0.5 mg/ml to about 16 mg/ml in a water, wherein the solution has at least one of the following characteristics:

(a) The sum of impurities in the solution at a gemcitabine concentration of about 10 mg/ml, a pH of about 8, and a temperature of about 25° C. for about 1 month is less than about 0.058% by area percent HPLC.

(b) The sum of impurities in the solution at a gemcitabine concentration of about 10 mg/ml, a pH of about 8, and a temperature of about 60° C. for about 1 month is less than about 0.381% by area percent HPLC.

(c) The sum of impurities in the solution at a gemcitabine concentration of about 10 mg/ml, a pH of about 8, and a temperature of about 25° C. for about 6 months is less than about 0.095% by area percent HPLC.

(d) The sum of impurities in the solution at a gemcitabine concentration of about 10 mg/ml, a pH of about 8, and a temperature of about 60° C. for about 6 months is less than about 2.745% by area percent HPLC.

(e) The sum of impurities in the solution at a gemcitabine concentration of about 10 mg/ml, a pH of about 8, and a temperature of about 25° C. for about 36 months is less than about 0.521% by area percent HPLC.

In a preferred embodiment, the solution has all of the characteristics (a) to (e).

### DETAILED DESCRIPTION OF THE INVENTION

The term “ready-to-use” means that the solution referred to is not reconstituted from a solid, such as, for example, from a crystalline or amorphous solid or a lyophilisate immediately before its administration to a mammal.

The term “gemcitabine solution,” unless otherwise specified, refers to solutions prepared according to the invention.

Experimental results indicate that ready-to-use gemcitabine solutions with a pH of about 3.5 to about 10 have high storage stability rates at various storage temperatures.

In addition to high storage stability rates, gemcitabine solutions prepared according to the invention with a pH below 4 do not produce the side-effects of intravenous administration such as infusion pain and local intolerance. The intravenous and locally better-tolerated gemcitabine solutions according to the invention are thus advantageous for the patient and attending physician along the lines of improved therapy acceptance and therapy compliance, especially in the case of cytostatic therapy, which has known side-effects caused by the active substance.

In one aspect of the invention, the present invention provides a gemcitabine solution with a pH of about 3.5 to about 10. Preferably, the solution has a pH of about 7 to about 10. More preferably, the pH is about 7 to about 9. Even more preferably, the pH of the solution is about 7.8 to about 8.2.

According to another aspect of the invention, the pH of the solution is about the same as the physiological pH of the tissues and blood, or about 7.35 to about 7.55.

In another aspect, the gemcitabine solution has a pH of about 4 to about 5.

The gemcitabine solution may contain about 0.5 mg to about 16 mg of gemcitabine per ml of solvent. Preferably, the gemcitabine concentration is about 10 mg/ml.

The gemcitabine solutions may be prepared from a free gemcitabine base, or a physiologically-acceptable acid addition salt thereof. Preferably, free gemcitabine base is used. More preferably, the acid addition salt of the gemcitabine base with an inorganic acid is used. Even more preferably, gemcitabine hydrochloride is used.

Suitable solvents for the gemcitabine solutions are, for example, water, ethyl alcohol, glycerine, 1,2-propanediol (propylene glycol), polyethylene glycol 200-600, benzyl alcohol, trimethylene glycol, 1,3-butylene glycol, 2,3-butylene glycol, ethyl acetate, ethyl lactate, glycofural (tetraglycol), solketal and urea. Preferably, water, ethyl alcohol, polyethylene glycol 200-600 or 1,2-propanediol (propylene glycol) are used. More preferably, water is used.

The pH of the gemcitabine solution can be adjusted by combining the gemcitabine base with a physiologically-acceptable acid addition salt thereof. Preferably, gemcitabine hydrochloride in suitable proportions is used.

The pH of the gemcitabine solution can also be adjusted with at least one physiologically-acceptable acidifier and/or alkalizer. Suitable acidifiers and/or alkalizers include, for example, inorganic acids and bases, such as, e.g., hydrochloric acid, sulfuric acid, sulfurous acid, nitric acid, nitrous acid, phosphoric acid, phosphorous acid, carbonic acid, sodium hydroxide, potassium hydroxide, calcium hydroxide and magnesium hydroxide; alkali and alkaline-earth salts as well as alkaline hydrogen salts and alkaline-earth hydrogen salts of the inorganic oxo salts of phosphorus, sulfur, carbon and nitrogen, such as, e.g., sodium phosphate and its hydrates, sodium dihydrogen phosphate and its hydrates, disodium hydrogen phosphate and its hydrates, disodium sulfate, sodium hydrogen sulfate, sodium sulfite, calcium sulfite, magnesium sulfite, calcium hydrogen carbonate, sodium carbonate, sodium hydrogen carbonate, sodium nitrate, sodium nitrite, calcium nitrite, magnesium nitrate, and magnesium nitrite; chlorine salts such as, e.g., sodium chloride, calcium chloride, and magnesium chloride; organic bases and acids, such as, e.g., formic acid, acetic acid, propionic acid, lactic acid, oxalic acid, malonic acid, maleic acid, tartaric acid, citric acid, pyruvic acid, benzoic acid, methanesulfonic acid, ethanesulfonic acid, para-toluenesulfonic acid, salicylic acid, ascorbic acid, tris(hydroxymethyl)aminomethane (2-amino-2-hydroxymethyl-1,3-propanediol); trometamol; TRIS), 1-deoxy-(methylamino)-D-glucitol (N-methylglucamine, meglumine); alkali salts and alkaline-earth salts of organic bases and acids, such as sodium acetate; and mixtures thereof.

Preferably, the pH is adjusted with hydrochloric acid, phosphoric acid, sulfuric acid, sodium hydroxide, sodium phosphate and its hydrates, sodium hydrogen phosphate and its hydrates, disodium hydrogen phosphate and its hydrates, acetic acid, lactic acid, citric acid, methanesulfonic acid, ethanesulfonic acid, tris(hydroxymethyl)aminomethane (trometamol; TRIS) or 1-deoxy-(methylamino)-D-glucitol (N-methylglucamine, meglumine). More preferably, the pH is adjusted with sodium hydroxide, hydrochloric acid, tris(hydroxymethyl)aminomethane (trometamol; TRIS) or 1-deoxy-(methylamino)-D-glucitol (N-methylglucamine, meglumine).

The pH can also be adjusted and/or stabilized by a buffer formed from a physiologically-acceptable acidifier and/or alkalizer. Preferably, buffer systems where the pK of at least one functional group of the buffer base or buffer acid is about 2.49 to about 11.01 is used. More preferred buffers are sodium acetate, tris(hydroxymethyl)aminomethane (trometamol; TRIS), 1-deoxy-(methylamino)-D-glucitol (N-methylglucamine, meglumine), disodium hydrogen phosphate, and mixtures thereof.

The concentration of the buffer is about 0.001 g to about 100 g buffer component per 1 g gemcitabine. Preferably, about 0.05 g to about 20 g buffer component per 1 g gemcitabine. More preferably, about 0.1 g to about 10 g buffer component per 1 g gemcitabine.

The gemcitabine solutions may optionally contain, in addition, at least one tonic adjuvant, preservative, and/or antioxidant.

For example, the gemcitabine solution may contain the following tonic agents: physiologically-acceptable inorganic alkali or alkaline-earth salts, such as, e.g., sodium chloride, calcium chloride, magnesium chloride, sodium sulfate, sodium carbonate and calcium hydrogen carbonate; physiologically-acceptable organic salts, such as, e.g., sodium lactate; physiologically-acceptable carbohydrates, such as, e.g., physiologically-acceptable cyclodextrin (α-, β-, γ-cyclodextrin), as well as their alkyl-substituted and/or aryl-substituted derivatives, glucose, fructose, sorbitol, mannitol, galactose, inositol, maltitol, lactose, trehalose, maltose, sucrose, dextran 1, dextran 10, dextran 40, dextran 70, starch, and hydroxyethyl starch; physiologically-acceptable amino acids, peptides or proteins, such as, e.g., glycine, albumin and gelatins, and mixtures thereof.

Preferred tonic agents are sodium chloride, calcium chloride, glucose, mannitol, and lactose. More preferred tonic agents are sodium chloride, glucose, and mannitol.

Preservatives may include, for example, chlorocresol, benzyl alcohol, p-hydroxybenzoic acid ester, ethylparaben, and methylparaben. Preferred preservatives are benzyl alcohol, propylparaben and methylparaben. A more preferred preservative is benzyl alcohol.

Antioxidants may include, for example, oxygen-reducing antioxidants, such as, for instance, sodium metabisulfite and sodium sulfite, or transition-metal-binding antioxidants and complexing (chelating) antioxidants, such as sodium edetate.

Optionally, at least one other drug solution may be admixed to the ready-to-use gemcitabine solution. For example, the gemcitabine solution can be admixed with a cisplatin solution to obtain a cytostatic infusion cocktail.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

To determine the solubility of gemcitabine in aqueous solutions (at T=25° C. and T=2-8° C., initial pH=4.0), gemcitabine hydrochloride was dissolved in water for injection at 25° C. (gemcitabine concentration of 55.2 mg/ml corresponding to 62.8 mg of gemcitabine hydrochloride per ml of water as well as gemcitabine concentration of 10.0 mg/ml corresponding to 11.38 mg of gemcitabine hydrochloride per ml of water). The pH was adjusted with q.s. of 1N sodium hydroxide solution/q.s. of 1N hydrochloric acid.

Dilute solutions were prepared from the batch thus obtained with water for injection and their pH was determined potentiometrically. The prepared solutions were each filled as 5-mL aliquots, into vials tightly sealed with perforable stoppers. These samples, after storage at 2-8° C. or 25° C., were checked for visible crystallization of the active substance (“+” precipitate; “−” clear and free of particles), and their pH was determined.

TABLE 1
Precipitation in gemcitabine solutions stored at 25° C. and 2-8° C.
(initial pH about 4.0)

| | Gemcitabine | | | | | | | |
|---|---|---|---|---|---|---|---|---|
| | Base | Final | After 3 d | After 1 d | After 2 d | After 3 d | After 7 d | After 14 d |
| | (mg/ml) | pH | (25° C.) | (2-8° C.) | (2-8° C.) | (2-8° C.) | (2-8° C.) | (2-8° C.) |
| 1 | 55.2 | 1.78 | − | + | + | + | + | + |
| 2 | 55.2 | 3.98 | − | ++++ | ++++ | ++++ | ++++ | ++++ |
| 3 | 46.0 | 4.14 | − | ++++ | ++++ | ++++ | ++++ | ++++ |
| 4 | 41.4 | 4.18 | − | ++++ | ++++ | ++++ | ++++ | ++++ |
| 5 | 36.8 | 4.20 | − | ++++ | ++++ | ++++ | ++++ | ++++ |
| 6 | 32.2 | 4.23 | − | +++ | +++ | +++ | +++ | +++ |
| 7 | 27.6 | 4.25 | − | ++ | ++ | ++ | ++ | ++ |
| 8 | 23.0 | 4.27 | − | + | + | + | + | + |
| 9 | 18.4 | 4.29 | − | − | + | + | + | + |
| 10 | 13.8 | 4.32 | − | − | + | + | + | + |
| 11 | 10.0 | 3.97 | − | − | − | − | − | − |
| 12 | 9.0 | 3.99 | − | − | − | − | − | − |
| 13 | 8.0 | 4.00 | − | − | − | − | − | − |
| 14 | 7.0 | 4.01 | − | − | − | − | − | − |
| 15 | 6.0 | 4.02 | − | − | − | − | − | − |
| 16 | 5.0 | 4.03 | − | − | − | − | − | − |
| 17 | 4.0 | 4.04 | − | − | − | − | − | − |
| 18 | 3.0 | 4.06 | − | − | − | − | − | − |
| 19 | 2.0 | 4.08 | − | − | − | − | − | − |
| 20 | 1.0 | 4.10 | − | − | − | − | − | − |

The data listed in Table 1 shows that clear, particle-free gemcitabine solutions containing about 10 mg/ml of gemcitabine (about 11.4 mg of gemcitabine hydrochloride per ml of water) with a pH of about 4 stored at 2-8° C. could be obtained without the additional use of solubility-enhancing auxiliary agents.

### Example 2

The information for use and technical data of GEMZAR® lyophilisate indicate that, when it is reconstituted with NaCl solution the pH obtained is within the range of 2.7 to 3.3. The solutions thus obtained, which must be prepared from the solid substance immediately before administration to the patient, can be stored only at 25° C., but not (short-term) at temperatures of 2-8° C. Moreover, it is generally known from the state of the art that the solubility of gemcitabine in water decreases with increasing pH.

To determine the actual pH-dependent solubility of gemcitabine in aqueous solutions, concentrated gemcitabine solutions (c.=52.7 mg of gemcitabine/ml corresponding to 60 mg of gemcitabine hydrochloride per ml of water in 5 ml/vial) were prepared according to Example 1, with the pH being adjusted to levels of 2.0 to 5.0 with q.s. of 1N sodium hydroxide. The solutions thus obtained were stored at a temperature of 2-8° C.

TABLE 2
Stability of concentrated gemcitabine solutions vs. Crystallization of the
active substance

| Visible | | | | | | | |
|---|---|---|---|---|---|---|---|
| Crystallization | | | | | | | |
| c. = 52.7 mg | | | | | | | |
| gemcitabine/ml | pH 2.0 | pH 2.5 | pH 3.0 | pH 3.5 | pH 4.0 | pH 4.5 | pH 5.0 |
| After 1 day (2-8° C.) | + | − | − | − | +++ | ++++ | ++++ |
| After 2 days (2-8° C.) | + | + | + | − | +++ | ++++ | ++++ |
| After 3 days (2-8° C.) | + | + | + | − | +++ | ++++ | ++++ |
| After 7 days (2-8° C.) | + | + | + | − | +++ | ++++ | ++++ |
| After 14 days (2-8° C.) | + | + | + | − | +++ | ++++ | ++++ |

These experiments showed that gemcitabine solutions at a pH beyond the range of about pH 3.5 exhibited a visible undesired and partly spontaneous (pH of 5.0; 25° C.) crystallization (“+”=precipitate; “−”=clear and particle-free).

The data listed in Table 2 indicate that without the additional use of solubility-enhancing auxiliary substances, concentrated gemcitabine solutions stored at 2-8° C. remain clearly dissolved, free of visible particles, only within a very narrow acid pH range.

To further determine the temperature dependence of the stability of gemcitabine solutions according to the invention with a pH of about 4.0, solutions as described above, with pH adjusted with 1N sodium hydroxide, were prepared with a gemcitabine concentration of 10 mg/ml (in water for injection). The solutions were stored for a period of 12 months. During this time, the concentration of active substance was determined at regular intervals by HPLC.

TABLE 3
Temperature-dependent stability of gemcitabine solutions (c. of
gemcitabine = 10 mg/ml, corresponding to 11.39 mg
gemcitabine hydrochloride per ml of water, pH 4.0)

| | 5° C. | 15° C. | 25° C. |
|---|---|---|---|
| | Content | Content | Content |
| Test Action | (rel. %) | (rel. %) | (rel. %) |
| 0 month | 100.0 | 100.0 | 100.0 |
| 3 months | 99.7 | 99.6 | 97.1 |
| 6 months | 99.4 | 99.2 | 94.1 |
| 9 months | 99.2 | 98.8 | — |
| 12 months | 99.0 | 98.3 | — |

Sum of Imp. = Sum of impurities as determined by HPLC.

At temperatures of up to 15° C., the solutions tested exhibited pharmaceutically-acceptable storage stability.

### Example 3

To determine the pH-dependence of the stability of buffered gemcitabine solutions, gemcitabine solutions (c.=38 mg/ml) with selected pH values within a range of 3 through 10 were subjected to stability studies. Solutions were prepared, each containing 10 mg/ml of gemcitabine (in terms of the base), 10 mg/ml of mannitol, 5.0 mg/ml of sodium chloride and water for injection, q.s. ad 1 ml of solution. The pH was adjusted by the addition of the required amounts of 1N sodium hydroxide or 1N hydrochloric acid in each case. In addition, the buffers listed in the following Table 4 were used to stabilize the pH in each instance. The pH levels were determined potentiometrically.

TABLE 4
Gemcitabine solutions - pH and buffers used

| Test Solution | pH | Buffer |
|---|---|---|
| 1 | 3 | 0.625 mg/ml sodium acetate |
| 2 | 4 | 0.625 mg/ml sodium acetate |
| 3 | 5 | 0.625 mg/ml sodium acetate |
| 4 | 6 | 0.625 mg/ml sodium acetate + 10 μmol/ml |
| | | disodium hydrogen phosphate, dihydrate |
| 5 | 7 | 0.625 mg/ml sodium acetate + 10 μmol/ml |
| | | disodium hydrogen phosphate, dihydrate |
| 6 | 8 | 0.625 mg/ml sodium acetate + 10 μmol/ml |
| | | disodium hydrogen phosphate, dihydrate |
| 7 | 9 | 0.625 mg/ml sodium acetate + 10 μmol/ml |
| | | N-methylglucamine |
| 8 | 10 | 0.625 mg/ml sodium acetate + 10 μmol/ml |
| | | N-methylglucamine |

To produce the above solutions, 80% of the required water for injection was prepared in advance. The ingredients required for each batch, with the exception of the active substance, were added one after another and dissolved. Then the gemcitabine hydrochloride was added and dissolved until a clear solution was obtained. The desired pH was adjusted with the required amounts of 1N sodium hydroxide or 1N hydrochloric acid (with a maximum deviation of ±0.2 per pH level). The pH was determined potentiometrically. The batch was made up with water for injection and, when necessary, the pH was again adjusted with the required amounts of 1N sodium hydroxide or 1N hydrochloric acid (maximum deviation of ±0.2 per pH level).

Each of these 50-mL test solutions were sterilized by filtration, and each 2 ml filled aseptically into a 2-mL perforable stopper vial. The vials were sealed with perforable stoppers, crimped airtight, and stored at 55° C. for a period of one month “under accelerated conditions.”

The “pH” stability parameter was determined potentiometrically and the “content and purity” stability parameters were determined by HPLC.

The results of the studies undertaken “under accelerated conditions” at 55° C. are listed in Table 5 below. “Stability of the solution” is always understood to refer to the stability of the gemcitabine in solution, i.e., as the long-term constant concentration of the starting compound after being reconstituted in solution.

TABLE 5
Stability data of gemcitabine solutions at pH 3-10 and 55° C.

| | | | | | | 1 |
|---|---|---|---|---|---|---|
| | | Starting | 1 Month | 1 Month | Starting | Month |
| | | Gemcitabine | Gemcitabine | Content | Sum | Sum |
| Test | | Content | Content | Loss¹ | of Imp.² | of Imp. |
| Solution | pH | [rel. in %] | [rel. in %] | [rel. in %] | [%] | [%] |
| 1 | 3 | 100.0 | 60.2 | −39.8 | 0.030 | 18.770 |
| 2 | 4 | 100.0 | 84.5 | −15.5 | 0.025 | 8.353 |
| 3 | 5 | 100.0 | 94.0 | −6.0 | 0.025 | 1.459 |
| 4 | 6 | 100.0 | 99.4 | −0.6 | 0.026 | 0.559 |
| 5 | 7 | 100.0 | 99.1 | −0.9 | 0.027 | 0.478 |
| 6 | 8 | 100.0 | 99.3 | −0.7 | 0.027 | 0.475 |
| 7 | 9 | 100.0 | 97.0 | −3.0 | 0.027 | 0.476 |
| 8 | 10 | 100.0 | 97.0 | −3.0 | 0.035 | 1.138 |

¹Content loss of gemcitabine relative to the starting value.
²Sum Imp. = Sum of impurities.

The pH of the test solutions with a pH of 5-10 remained unchanged over the period of the accelerated stability study, with a maximum tolerance of ±0.2 pH level. The test solutions with a pH of 3 and 4 showed a pH increase of up to one pH level, which correlates with the degree of degradation of the active substance in the test solutions.

The data listed in Table 5 of the experimental series show that the optimum stability of gemcitabine in aqueous solution (c.=10 mg/ml) is not in the strongly acidic pH range, but in the range of pH 4 to 10, preferably at pH 6 to 9, more preferably at pH 7 to 9, and even more preferably at a pH of about 8.

Compared to the stability of conventional infusion solutions with pH values of about 3 (2.7 to 3.3), the degradation of gemcitabine, in a pH range of about 4.0 to about 10.0, can be reduced by about 3- to 57-fold and an increase of the sum of impurities can be reduced by about 13- to 40-fold.

### Example 4

A buffered gemcitabine solution of the composition indicated below (test solution 9), with a pH of about 8.0 (7.8-8.2), was subjected to a stability study.

Composition of Test Solution 9:

| | |
|---|---|
| Gemcitabine | 10.0 mg/ml (in term of the base) |
| Mannitol | 10.0 mg/ml |
| Sodium acetate | 0.625 mg/ml |
| Sodium chloride | 5.0 mg/ml |

1N sodium hydroxide or possibly 1N hydrochloric acid q.s. ad pH 8.0
Water for injection q.s. ad 1 ml of solution.

To produce the above solution (test solution 9), about 80% of the water for injection required (at 20-25° C.) was transferred to the batch container beforehand. Mannitol, sodium acetate, and sodium chloride were added one after another and dissolved until a clear solution was obtained. Then gemcitabine hydrochloride was added and dissolved until a clear solution was obtained. The desired pH was adjusted with the required amounts of 1N sodium hydroxide or 1N hydrochloric acid (maximum deviation: ±0.2 pH level). The pH was determined potentiometrically. The batch was filled up with water for injection and, when necessary, the pH again adjusted with the required amounts of 1N sodium hydroxide or 1N hydrochloric acid (deviation: ±0.2 pH level).

500-mL of this test solution was sterilized by filtration, each 2 ml filled aseptically into a 2-mL perforable stopper vial. The were vials sealed with pierce-through stoppers, crimped airtight, and stored protected from light at 25° C., 40° C. and 60° C. for a period of 18 months.

The “pH” stability parameter was determined potentiometrically and the “content and purity” stability parameters were determined by HPLC.

Table 6 illustrates the results from the study undertaken at 25° C., 40° C., and 60° C. “under accelerated conditions” to determine the storage stability of gemcitabine solutions.

TABLE 6
Stability of gemcitabine solutions at pH of 8 and 25° C.,
40° C. and 60° C.

| | 25° C. | 25° C. | 40° C. | 40° C. | | 60° C. |
|---|---|---|---|---|---|---|
| | Content | Sum of | Content | Sum of | 60° C. | Sum of |
| | [rel. | Imp.* | [rel. | Imp.* | Content | Imp.* |
| | in %] | [%] | in %] | [%] | [rel. in %] | [%] |
| Start | 100.0 | 0.040 | 100.0 | 0.040 | 100.0 | 0.040 |
| 1 month | 100.01 | 0.058 | 100.02 | 0.104 | 99.62 | 0.381 |
| 2 months | 100.04 | 0.069 | 100.02 | 0.174 | 98.41 | 0.938 |
| 3 months | 99.79 | 0.076 | 100.04 | 0.216 | 97.53 | 1.217 |
| 6 months | 99.61 | 0.095 | 97.22 | 0.439 | 94.79 | 2.745 |
| 9 months | 99.23 | 0.184 | 98.83 | 0.668 | — | — |
| 12 months | 99.55 | 0.196 | 98.31 | 0.951 | — | — |
| 18 months | 99.02 | 0.279 | — | — | — | — |

*Sum Imp. = Sum of impurities.

At a storage temperature of 25° C. for a period of 18 months, the solutions remained clear, colorless, and free of visible particles or crystal formation.

The pH remained almost unchanged over the period of the stability study with a maximum tolerance of ±0.2 pH level (at 25° C.) or ±0.3 pH level (at 55° C.).

A regressive extrapolation of the data listed in Table 6 yielded the following storage stability data indicated in Table 7 below.

TABLE 7
Stability of gemcitabine solutions at pH 8 and 25° C., 40° C. and 60° C.
during a storage period of 1-4 years

| | 25° C. | 25° C. | 40° C. | 40° C. | 60° C. | |
|---|---|---|---|---|---|---|
| | Content | Sum of | Content | Sum of | Content | 60° C. Sum of |
| | [rel. in %] | Imp.* [%] | [rel. in %] | Imp.* [%] | [rel. in %] | Imp.* [%] |
| 12 months | 99.34 | 0.200 | 97.92 | 0.916 | 89.43 | 5.415 |
| 24 months | 98.67 | 0.361 | 95.80 | 1.818 | 78.64 | 10.85 |
| 36 months | 97.99 | 0.521 | 93.68 | 2.719 | 67.85 | 16.29 |
| 48 months | 97.32 | 0.682 | 91.56 | 3.621 | 57.05 | 21.73 |

*Sum Imp. = Sum of impurities.

The data show that, at a concentration of 10 mg/ml, gemcitabine in aqueous solution at a pH of about 8 exhibits high storage stability at a storage temperature of 25° C. Furthermore, these solutions are stable for at least 3 years at a storage temperature of 25° C.

### Example 5

To determine the concentration dependence of the stability of the gemcitabine solutions, the solutions indicated below with a pH of about 8.0 (7.8-8.2) were prepared and analyzed.

Test Solutions 10-12:

Solutions with

0.05 mg/ml (=50 μg/ml) of gemcitabine base

0.25 mg/ml (=250 μg/ml) of gemcitabine base

1.00 mg/ml of gemcitabine base

were prepared with q.s. of water for injection for 1 ml. The pH was adjusted to about 8.0 (7.8-8.2) with q.s. of hydrochloric acid or sodium hydroxide and stabilized with 2.0 mg/ml of trometamol.

The required amounts of gemcitabine were dissolved in 80% of the required water for injection and the trometamol was then dissolved therein. The pH was adjusted to about 8.0 (±0.2) with q.s. of 1N hydrochloric acid or 1N sodium hydroxide. The batches were filled [up to the mark] with water for injection and, when necessary, the pH was readjusted to 8.0 (±0.2), as described above. The batches were sterilized by filtration, each vial filled with 5 ml aseptically, and the vials sealed airtight with injection stoppers and crimping caps.

The batches were stored protected from light at 40° C. (under “accelerated conditions”). The gemcitabine concentration was determined by HPLC.

The pH was determined potentiometrically. The deviations during the test period were ±0.2 pH level. The results of the studies are listed in Table 8 below.

TABLE 8
Dependence of the stability of gemcitabine solutions on the
concentration

| Gemcitabine | Gemcitabine | Gemcitabine |
|---|---|---|
| Concentration | Concentration | Concentration |
| 0.05 mg/ml | 0.25 mg/ml | 1.00 mg/ml |
| Start: 100% | Start: 100% | Start: 100% |
| 1 month: 99.8% | 1 month: 100.0% | 1 month: 100.0% |
| 3 months: 99.3% | 3 months: 99.5% | 3 months: 99.7% |
| 6 months: 98.3% | 6 months: 98.8% | 6 months: 99.3% |

As shown in Table 8, the gemcitabine solutions are stable in storage, even at concentrations down to c.=0.05 mg/ml.

### Examples 6-18

To determine the stability of pH-adjusted gemcitabine solutions according to the invention and partly containing other adjuvants, preservatives and antioxidants, corresponding solutions were prepared as described above, stored for 12 months, and the concentration of the active substance was determined by HPLC. From these data it was possible to interpolate or extrapolate the expected storage time (t₉₅) during which an active substance content of 95% of the starting concentration was still present.

The results of these studies are listed in Table 9.

TABLE 9
t₉₅ - Stability data of prepared gemcitabine solutions

| | Example: Weighed in [mg/ml] | | | | | | | | | | | | |
|---|---|---|---|---|---|---|---|---|---|---|---|---|---|
| | 6 | 7 | 8 | 9 | 10 | 11 | 12 | 13 | 14 | 15 | 16 | 17 | 18 |
| Gemcitabine | 10.0 | 10.0 | 10.0 | 10.0 | 10.0 | 10.0 | 10.0 | 10.0 | 10.0 | 10.0 | 10.0 | 10.0 | 10.0 |
| 1N NaOH/ | q.s. | q.s. | q.s. | q.s. | q.s. | q.s. | q.s. | q.s. | q.s. | q.s. | q.s. | q.s. | q.s. |
| 1N HCl | | | | | | | | | | | | | |
| NaCl | 6.50 | — | — | 6.50 | 5.50 | 3.50 | 6.50 | 3.50 | 4.00 | 5.00 | — | 5.0 | — |
| Mannitol | — | 40.0 | — | — | — | — | — | — | — | 10.0 | — | — | — |
| Glucose.H₂0 | — | — | 45.0 | — | — | — | — | — | — | — | — | — | — |
| Trometamol | — | — | — | 0.25 | — | — | — | — | — | — | — | — | — |
| Sodium | — | — | — | — | — | 2.00 | — | 2.00 | — | — | — | — | — |
| metabisulfite | | | | | | | | | | | | | |
| Sodium edetate | — | — | — | — | — | — | 0.10 | 0.10 | — | — | — | — | — |
| Trometamol | — | — | — | — | — | 2.00 | — | 2.00 | 10.0 | — | — | — | — |
| Sodium acetate | — | — | — | — | — | — | — | — | — | 0.625 | — | — | 3.25 |
| Methylparaben | — | — | — | — | 1.30 | — | — | — | — | — | — | — | — |
| Propylparaben | — | — | — | — | 0.20 | — | — | — | — | — | — | — | — |
| Benzyl alcohol | — | — | — | — | — | — | — | — | 10.0 | — | — | — | — |
| Water for | q.s. | q.s. | q.s. | q.s. | q.s. | q.s. | q.s. | q.s. | q.s. | q.s. | q.s. | q.s. | q.s. |
| injection | | | | | | | | | | | | | |
| pH | 8.1 | 8.2 | 8.0 | 8.0 | 7.8 | 7.9 | 8.0 | 8.1 | 7.9 | 8.3 | 3.9 | 4.1 | 3.5 |
| t₉₅* (mo.; 2-8° C.) | — | — | — | — | — | — | — | — | — | — | 56 | 52 | 17 |
| t₉₅* (mo.; 15° C.) | — | — | — | — | — | — | — | — | — | — | 36 | 28 | 2 |
| t₉₅* (mo.; 25° C.) | 108 | 100 | 85 | 77 | 110 | 82 | 57 | 87 | 91 | 146 | 5 | 4 | 2 |
| t₉₅* (mo.; 40° C.) | 20 | 20 | 18 | 17 | 19 | 18 | 16 | 17 | 19 | 22 | — | — | 0.5 |
| t₉₅* (mo.; 60° C.) | 6 | 7 | 6 | 5 | 6 | 5 | 4 | 6 | 5 | 6 | — | — | — |

*rounded

The stability data indicate that gemcitabine solutions according to the invention (with a pH of about 8), stored at 2-8° C. (pH of about 4) as well as at 25° C., are stable for a period of at least 3 years.

### Examples 19-63

Aqueous solutions of the following composition were prepared:

| | Gemcitabine | Alkalizer | | | |
|---|---|---|---|---|---|
| | Content | or | | Buffer | |
| Example | [mg/ml] | Acidifier | pH | [m/V] | Adjuvant [m/V] |
| 19 | 0.06 | TRIS | 8.2 | q.s. TRIS | — |
| 20 | 1.0 | Sodium | 8.3 | | 0.9% NaCl |
| | | hydroxide | | | |
| 21 | 5.0 | Sodium | 8.7 | | 0.9% NaCl |
| | | hydroxide | | | |
| 22 | 5.0 | Sodium | 8.7 | 0.05% | 0.9% NaCl |
| | | hydroxide | | Meglumine | |
| 23 | 10.0 | TRIS | 5.0 | 0.02% Sodium | 5% Mannitol |
| | | | | dihydrogen | |
| | | | | phosphate | |
| 24 | 10.0 | Meglumine | 8.0 | | 5% Glucose |
| 25 | 16.0 | Sodium | 8.0 | | |
| | | hydroxide/ | | | |
| | | Hydrochloric acid | | | |
| 26 | 10.0 | Sodium | 8.0 | | 0.65% |
| | | hydroxide/ | | | Sodium |
| | | Hydrochloric | | | chloride |
| | | acid | | | |
| 27 | 10.0 | Sodium | 8.0 | | 4.0% Mannitol |
| | | hydroxide/ | | | |
| | | Hydrochloric | | | |
| | | acid | | | |
| 28 | 10.0 | Sodium | 8.0 | | 4.5% Glucose |
| | | hydroxide/ | | | |
| | | Hydrochloric | | | |
| | | acid | | | |
| 29 | 10.0 | Sodium | 8.0 | 0.025% | 0.65% |
| | | hydroxide/ | | Trometamol | Sodium |
| | | Hydrochloric | | | chloride |
| | | acid | | | |
| 30 | 10.0 | Sodium | 8.0 | | 0.55% |
| | | hydroxide/ | | | Sodium |
| | | Hydrochloric | | | chloride, |
| | | acid | | | 0.13% |
| | | | | | Methylparaben, |
| | | | | | 0.02% |
| | | | | | Propylparaben |
| 31 | 10.0 | Sodium | 8.0 | 0.5% | 0.55% |
| | | hydroxide/ | | Trometamol | Sodium |
| | | Hydrochloric | | | chloride, |
| | | acid | | | 0.20% |
| | | | | | Sodium |
| | | | | | metabisulfite |
| 32 | 10.0 | Sodium | 8.0 | | 0.65% |
| | | hydroxide/ | | | Sodium |
| | | Hydrochloric | | | chloride, |
| | | acid | | | 0.01% |
| | | | | | Sodium |
| | | | | | edetate |
| 33 | 10.0 | Sodium | 8.0 | 0.5% | 0.25% |
| | | hydroxide/ | | Trometamol | Sodium |
| | | Hydrochloric | | | chloride, |
| | | acid | | | 0.20% |
| | | | | | Sodium |
| | | | | | metabisulfite, |
| | | | | | 0.01% |
| | | | | | Sodium |
| | | | | | edetate |
| 34 | 10.0 | Sodium | 8.0 | | 0.40% |
| | | hydroxide/ | | | Sodium |
| | | Hydrochloric | | | chloride, |
| | | acid | | | 1.0% |
| | | | | | Benzyl alcohol |
| 35 | 10.0 | Meglumine | 9.0 | | 0.70% |
| | | | | | Sodium |
| | | | | | chloride |
| 36 | 10.0 | Meglumine | 10.0 | | 0.70% |
| | | | | | Sodium |
| | | | | | chloride |
| 37 | 10.0 | Disodium | 8.0 | | 0.65% |
| | | hydrogen | | | Sodium |
| | | phosphate, | | | chloride |
| | | dihydrate | | | |
| 38 | 16.0 | Sodium | 10.0 | | |
| | | hydroxide | | | |
| 39 | 16.0 | Sodium | 5.0 | | |
| | | hydroxide | | | |
| 40 | 16.0 | Sodium | 5.0 | 0.0625% | |
| | | hydroxide | | Sodium acetate | |
| 41 | 0.05 | Sodium | 10.0 | 0.2% | |
| | | hydroxide/ | | Meglumine | |
| | | Hydrochloric | | | |
| | | acid | | | |
| 42 | 0.05 | Meglumine | 10.0 | | |
| 43 | 0.05 | Meglumine | 9.0 | | 0.90% |
| | | | | | Sodium |
| | | | | | chloride |
| 44 | 0.05 | Trometamol | 8.0 | | |
| 45 | 16.0 | Sodium | 10.0 | | |
| | | hydroxide/ | | | |
| | | Hydrochloric | | | |
| | | acid | | | |
| 46 | 10.0 | Sodium | 4.0 | | |
| | | hydroxide/ | | | |
| | | Hydrochloric | | | |
| | | acid | | | |
| 47 | 10.0 | Sodium | 4.0 | | |
| | | hydroxide/ | | | |
| | | Lactic acid | | | |
| 48 | 10.0 | Sodium | 4.0 | | |
| | | hydroxide/ | | | |
| | | Lactic acid | | | |
| 49 | 10.0 | Sodium | 5.0 | | |
| | | hydroxide/ | | | |
| | | Acetic acid | | | |
| 50 | 10.0 | Sodium | 4.0 | 0.05% | |
| | | hydroxide/ | | Sodium citrate | |
| | | Hydrochloric | | | |
| | | acid | | | |
| 51 | 10.0 | Sodium | 4.0 | 0.05% | |
| | | hydroxide/ | | Sodium citrate | |
| | | Lactic acid | | | |
| 52 | 5.0 | Sodium | 4.0 | 0.04% | 0.55% |
| | | hydroxide/ | | Sodium lactate | Sodium |
| | | Lactic acid | | | chloride |
| 53 | 5.0 | Sodium | 5.0 | 0.05% | 0.55% |
| | | hydroxide/ | | Sodium acetate | Sodium |
| | | Acetic acid | | | chloride |
| 54 | 5.0 | Sodium | 4.0 | | 0.55% |
| | | hydroxide/ | | | Sodium |
| | | Hydrochloric | | | chloride |
| | | acid | | | |
| 55 | 5.0 | Sodium | 4.0 | | 0.60% |
| | | hydroxide/ | | | Sodium |
| | | Lactic acid | | | chloride |
| 56 | 5.0 | Sodium | 4.0 | | 0.55% |
| | | hydroxide/ | | | Sodium |
| | | Lactic acid | | | chloride |
| 57 | 5.0 | Sodium | 4.0 | | 0.60% |
| | | hydroxide/ | | | Sodium |
| | | Acetic acid | | | chloride |
| 58 | 0.5 | Sodium | 4.0 | | |
| | | hydroxide/ | | | |
| | | Acetic acid | | | |
| 59 | 0.05 | Sodium | 4.0 | | 0.90% |
| | | hydroxide/ | | | Sodium |
| | | Acetic acid | | | chloride |
| 60 | 0.05 | Sodium | 4.0 | | |
| | | hydroxide/ | | | |
| | | Acetic acid | | | |
| 61 | 10.0 | Sodium | 3.5 | | |
| | | hydroxide/ | | | |
| | | Acetic acid | | | |
| 62 | 1.0 | Sodium | 3.5 | | |
| | | hydroxide/ | | | |
| | | Acetic acid | | | |
| 63 | 0.05 | Sodium | 3.5 | | |
| | | hydroxide/ | | | |
| | | Acetic acid | | | |

What is claimed is:

1. An injectable pharmaceutical composition comprising a solution of gemcitabine having a pH of about 3.5 to about 10 and a gemcitabine concentration of about 0.5 mg/ml to about 16 mg/ml in a solvent.

2. The pharmaceutical composition according to claim 1, wherein the composition is not reconstituted from a solid substance at least 24 hours before being administered to a mammal.

3. The pharmaceutical composition according to claim 2, wherein the composition is not reconstituted from a solid substance at least 72 hours before being administered to a mammal.

4. A package for distribution comprising the pharmaceutical composition according to claim 1, wherein the solution is ready for administration to a mammal without further solubilization of gemcitabine.

5. The pharmaceutical composition according to claim 1, wherein the gemcitabine concentration is about 5 mg/ml to about 12 mg/ml.

6. The pharmaceutical composition according to claim 5, wherein the gemcitabine concentration is about 10 mg/ml.

7. The pharmaceutical composition according to claim 1, wherein the solution is prepared from a free gemcitabine base.

8. The pharmaceutical composition according to claim 1, wherein the solution is prepared from a physiologically-acceptable acid addition salt of gemcitabine base.

9. The pharmaceutical composition according to claim 8, wherein the physiologically-acceptable acid addition salt is gemcitabine hydrochloride.

10. The pharmaceutical composition according to claim 1, wherein the solvent is selected from the group consisting of water, ethyl alcohol, polyethylene glycol 200-600, and propylene glycol.

11. The pharmaceutical composition according to claim 10, wherein the solvent is water.

12. The pharmaceutical composition according to claim 1, wherein the solution has a pH of about 7 to about 9.

13. The pharmaceutical composition according to claim 12, wherein the solution has a pH of about 7.8 to about 8.2.

14. The pharmaceutical composition according to claim 12, wherein the solution has a pH of about 7.35 to about 7.55.

15. The pharmaceutical composition according to claim 1, wherein the pH is adjusted by combining or converting gemcitabine base with/to a physiologically-acceptable acid addition salt thereof.

16. The pharmaceutical composition according to claim 15, wherein the physiologically-acceptable acid addition salt is gemcitabine hydrochloride.

17. The pharmaceutical composition according to claim 1, wherein the pH is adjusted with at least one physiologically-acceptable acid.

18. The pharmaceutical composition according to claim 17, wherein the acid is selected from the group consisting of hydrochloric acid, phosphoric acid, sulfuric acid, acetic acid, lactic acid, citric acid, methanesulfonic acid, and ethanesulfonic acid.

19. The pharmaceutical composition according to claim 18, wherein the acid is hydrochloric acid.

20. The pharmaceutical composition according to claim 1, wherein the pH is adjusted with at least one physiologically-acceptable base.

21. The pharmaceutical composition according to claim 20, wherein the base is selected from the group consisting of sodium hydroxide, potassium hydroxide, calcium hydroxide, and magnesium hydroxide.

22. The pharmaceutical composition according to claim 21, wherein the base is sodium hydroxide.

23. The pharmaceutical composition according to claim 1, further comprising a buffer.

24. The pharmaceutical composition according to claim 23, wherein at least one functional group of the buffer's acid or base is within the pK range about 2.5 to about 11.

25. The pharmaceutical composition according to claim 24, wherein the buffer is prepared from a reagent selected from the group consisting of tris(hydroxymethyl)-aminomethane, 1-deoxy-(methylamino)-D-glucitol, sodium acetate, disodium hydrogen phosphate, and mixtures thereof.

26. The pharmaceutical composition according to claim 23, wherein the buffer is in the amount of about 0.001 g to about 100 g buffer component per 1 g of gemcitabine.

27. The pharmaceutical composition according to claim 26, wherein the buffer is in the amount of about 0.05 g to about 20 g buffer component per 1 g of gemcitabine.

28. The pharmaceutical composition according to claim 27, wherein the buffer is in the amount of about 0.1 g to about 10 g buffer component per 1 g of gemcitabine.

29. The pharmaceutical composition according to claim 1, further comprising at least one tonic adjuvant, preservative, antioxidant, or mixtures thereof.

30. The pharmaceutical composition according to claim 1, wherein the solution has a gemcitabine concentration of about 10 mg/ml, a pH of about 8, and water as the solvent.

31. A method of treating tumors comprising administering the pharmaceutical composition according to claim 1 to a mammal in need thereof.

32. The pharmaceutical composition according to claim 1, wherein the solution remains free of visible particles after storage at a temperature of about 25° C. for about 18 months.

33. An injectable pharmaceutical composition comprising a solution of gemcitabine, a pH of about 3.5 to about 10, and a gemcitabine concentration of about 0.5 mg/ml to about 16 mg/ml in a water,
wherein the solution has at least one of the following characteristics:
(a) The sum of impurities in the solution at a gemcitabine concentration of about 10 mg/ml, a pH of about 8, and a temperature of about 25° C. for about 1 month is less than about 0.058% by area percent HPLC.
(b) The sum of impurities in the solution at a gemcitabine concentration of about 10 mg/ml, a pH of about 8, and a temperature of about 60° C. for about 1 month is less than about 0.381% by area percent HPLC.
(c) The sum of impurities in the solution at a gemcitabine concentration of about 10 mg/ml, a pH of about 8, and a temperature of about 25° C. for about 6 months is less than about 0.095% by area percent HPLC.
(d) The sum of impurities in the solution at a gemcitabine concentration of about 10 mg/ml, a pH of about 8, and a temperature of about 60° C. for about 6 months is less than about 2.745% by area percent HPLC.
(e) The sum of impurities in the solution at a gemcitabine concentration of about 10 mg/ml, a pH of about 8, and a temperature of about 25° C. for about 36 months is less than about 0.521% by area percent HPLC.

34. The pharmaceutical composition according to claim 33, wherein the solution has all of the characteristics (a) to (e).
